(19) United States
(12) Patent Application Publication
(10) Pub. No.: US 2026/0234701 A1
(43) Pub. Date: Aug. 13, 2026

(54) CRISPR TECHNOLOGIES FOR DIAGNOSTIC APPLICATIONS

(71) Applicants: UNIVERSITY HOSPITALS CLEVELAND MEDICAL CENTER, Cleveland, OH (US); CASE WESTERN RESERVE UNIVERSITY, Cleveland, OH (US)

(72) Inventor: Mohamed S. Draz, Cleveland, OH (US)

(21) Appl. No.: 19/153,017

(22) PCT Filed: Feb. 1, 2024
(86) PCT No.: PCT/US2024/014065

§ 371 Date: Aug. 1, 2025

Related U.S. Application Data
(60) Provisional application No. 63/482,651, filed on Feb. 1, 2023.

(51) Int. Cl. C12Q 1/6823; C12Q 1/34; C12Q 1/6834; C12Q 1/6844; C40B 70/00

(52) U.S. Cl. CPC C12Q 1/6823; C12Q 1/34; C12Q 1/6834; C12Q 1/6844; C12Y 301/00; C40B 70/00; C12Q 2600/112

(57) ABSTRACT

A system for detecting an analyte in a sample includes a plurality of barcode sequences that are configured to specifically bind to the analyte in the sample wherein each barcode sequence includes a nucleic acid and a reporter molecule and the nucleic acid of the barcode sequence is configured to be cleaved from the barcode sequence by a Cas nuclease effector protein.

### RELATED APPLICATION

This application claims priority from U.S. Provisional Application No. 63/482,651, filed Feb. 1, 2023, the subject matter of which is incorporated herein by in reference in its entirety.

### GOVERNMENT FUNDING

This invention was made with government support under TW012056 and CS254566 awarded by the National Institutes of Health. The government has certain rights in the invention.

### BACKGROUND

Current devices and systems for multiplex analyte testing typically require staining or multistep labeling that is necessarily performed using remote laboratory testing. Additionally, in current analyte sensing approaches that utilize color (e.g., ELISA) and fluorescence (e.g., PCR) labels, the generated color or fluorescence detection signal is generally diffused over a relatively large volume of sample that leads to lower detection sensitivity and sensing efficiency. Also, these current approaches may not enable efficient integration with AI-based image analysis systems or even simple optical systems with a camera. There remains a need for novel devices allowing for simpler methods and systems for rapid detection with high multiplexity, e.g., in point-of-care (POC) diagnostics, that can detect the target analyte with different modalities, such as color, fluorescence, etc., without the need for staining or multistep labeling.

### SUMMARY

Embodiments described herein relate to the use of CRISPR based technologies in a system for detecting an analyte in a sample, a diagnostic device of the analyte detection system, use of the analyte detection system as a diagnostic agent, a kit-of-parts for detecting an analyte, such as nucleic acids, a method for detecting an analyte, and a method for diagnosing a disease state of a subject. The detection system comprises a CRISPR-Cas system which include a Cas nuclease effector protein and one or more guide RNAs having a guide sequence. The guide sequence can be capable of targeting the effector protein to a target sequence of a target, such as a barcode sequence or target nucleic acid, and the effector protein can target-activated nucleic acid cleavage activity capable of cleaving reporter molecules that are indicative of the presence of the analyte in a sample.

In some embodiments, the system for detecting one or more analytes in a sample includes a plurality of magnetic beads, a plurality of analyte capture molecules extending from the surfaces of the beads, a CRISPR Cas nuclease effector protein, and a plurality of barcode sequences that are configured to specifically and selectively bind to the one or more analyte in the sample. Each barcode sequence includes a nucleic acid and a reporter molecule. The Cas nuclease effector protein is configured to cleave the nucleic acid of the barcode sequence and release the reporter molecule after binding of the barcode sequence to the analyte in the sample. The released reporter molecule can then be detected to indicate the presence of the analyte in the sample.

In some embodiments, the analyte is a targeted nucleic acid in a sample and the analyte capture molecule includes and/or defines the barcode sequence. The barcode sequence can include a single-stranded target-specific nucleic acid sequence that is complementary to or hybridizes with at least a portion of a targeted nucleic acid. The target-specific nucleic acid sequences of the barcode sequences can each include a first end linked to the magnetic bead and a second terminal end linked to the reporter molecule. The reporter molecule is configured to be cleaved from the barcode sequence by a Cas nuclease effector protein after binding of target-specific nucleic acid to the at least portion of the targeted nucleic acid.

In some embodiments, the Cas nuclease effector protein is complexed with a guide RNA sequence that includes a nucleotide sequence that hybridizes to or is complementary to the target-specific nucleic acid sequence or the portion of the targeted nucleic acid.

In some embodiments, the reporter molecule includes at least one of a dye including a fluorescent or a colorimetric dye, a marker molecule or polymer having a defined molecular weight, an electrically active or detectable marker, a hapten, enzyme fragment, co-factor or subunit, nanoparticle, radiolabel, contrast agent, quantum dots, and/or any other agent that is detectable by, for example, color detectors, turbidimeters, fluorometers, electrical signal detectors, lateral flow assays, electrophoresis, chromatography, and magnetic readers.

In other embodiments, the reporter molecule includes a small molecule that is degradable upon cleavage of the reporter molecule to release a plurality of detectable molecules indicative of the cleavage.

In other embodiments, the reporter molecule is a co-factor that activates an enzyme to generate color, fluorescence, or breakdown of a substrate into a detectable molecule.

In still other embodiments, the reporter molecule includes a DNA sequence that is readily amplified.

In some embodiments, the Cas nuclease effector protein comprises a Cas9, Cas12, or Cas13 nuclease.

In some embodiments, the target-specific nucleic acid of the barcode sequence is configured to target one or multiple genes of an organism or multiple organisms.

In some embodiments, the plurality of barcode sequences include first barcode sequences that include first target-specific nucleic acids and first reporter molecules and second barcode sequences that include second target-specific nucleic acids and second reporter molecules that differ from the first target-specific nucleic acids and first reporter molecules.

In other embodiments, the Cas nuclease includes a first Cas nuclease complexed with first guide RNA that is complementary or hybridizes to the first target specific nucleic acids or a first targeted nucleic acid and a second Cas nuclease complexed with a second guide RNA that is complementary or hybridizes to the second target-specific nucleic acids or a second targeted nucleic acid.

In some embodiments, the plurality of barcode sequences include barcode sequence having differing target specific nucleic acids and reporter molecules and the Cas nuclease includes Cas nuclease with differing guide RNAs that are complementary or hybridize to the differing target-specific nucleic acids or differing targeted nucleic acids.

In some embodiments, the plurality of barcode sequences are directly or indirectly linked to the plurality of magnetic beads.

In some embodiments, the plurality of barcode sequences are topographically and/or spatially arranged on an outer surface of the magnetic beads.

In some embodiments, the plurality of barcode sequences are linked to the magnetic beads using a click reaction chemistry.

In other embodiments, the analyte is a targeted polypeptide in the sample. The plurality of barcode sequences can include a nucleic acid with a first end and a second end. The first end can be linked to an antibody or antigen binding fragment thereof that specifically binds to the polypeptide, and the second end can include or be linked to a reporter molecule. The analyte capture molecules include a plurality of second antibody or antigen binding fragments thereof directly or indirectly linked to the magnetic beads. The second antibody or antigen binding fragments thereof can specifically bind to the polypeptide. The nucleic acid sequence of the barcode sequence is configured to be cleaved to release the reporter molecule from the barcode sequence by the Cas nuclease effector protein after binding of barcode sequence to the polypeptide.

In some embodiments, the Cas nuclease effector protein is complexed with a guide RNA sequence that includes nucleotide sequence that hybridizes or is complementary to at least a portion of the nucleic acid of the barcode sequence.

In some embodiments, the reporter molecule includes at least one of a dye including a fluorescent a colorimetric dye, a marker molecule or polymer having a defined molecular weight, an electrically active or detectable marker, a hapten, enzyme fragment, co-factor or subunit, nanoparticle, radiolabel, contrast agent, quantum dots, and/or any other agent that is detectable by, for example, color detectors, turbidimeters, fluorometers, electrical signal detectors, lateral flow assays, electrophoresis, chromatography, and magnetic readers.

In some embodiments the reporter molecule includes a small molecule that is degradable upon cleavage of the reporter molecule to release a plurality of detectable molecules indicative of the cleavage.

In some embodiments, the reporter molecule is a co-factor that activates an enzyme to generate color, fluorescence, or breakdown of a substrate into a detectable molecule.

In other embodiments, the reporter molecule includes a DNA sequence that is readily amplified.

In some embodiments, the Cas nuclease effector protein comprises a Cas9, Cas12, or Cas13 nuclease.

In some embodiments, the plurality of second antibodies or antigen binding fragments thereof are topographically and/or spatially arranged on an outer surface of the magnetic beads.

In some embodiments, the second antibody or antigen binding fragment thereof binds to a different epitope of the polypeptide than the first antibody or antigen binding fragment thereof of the barcode sequence.

Other embodiments described herein relate to a method of detecting an analyte in a sample. The method includes providing a plurality of barcode sequences that are configured to specifically bind to the analyte in the sample. Each barcode sequence can include a nucleic acid and reporter molecule. The nucleic acid of the barcode sequence is configured to be cleaved from the barcode sequence by a Cas nuclease effector protein after binding of barcode sequence to the analyte. The barcode sequences are combined with the sample such that the barcode binds to the analyte in the sample. The Cas nuclease effector protein can be added to the combined barcode sequences and sample, and the Cas nuclease effector protein can cleave the barcode sequences bound to the analyte to release the reporter molecule. The released reporter molecule can be detected or measured to detect the presence or quantity of the analyte in the sample.

In some embodiments, the analyte is a targeted nucleic acid and each barcode sequence includes a single-stranded target-specific nucleic acid sequence that is complementary to or hybridizes with at least a portion of the targeted nucleic acid and the reporter molecule is configured to be cleaved from the barcode sequence by the Cas nuclease effector protein after binding of target-specific nucleic acid to the at least portion of the targeted nucleic acid. In some embodiments, the Cas nuclease effector protein is complexed with a guide RNA sequence that includes nucleotide sequence that hybridizes to or is complementary to the target-specific nucleic acid sequence or the portion of the targeted nucleic acid.

In some embodiments, the released reporter molecule is detected to detect the presence of the nucleic acid in the sample.

In some embodiments, the reporter molecule includes at least one of a dye including a fluorescent a colorimetric dye, a marker molecule or polymer having a defined molecular weight, an electrically active or detectable marker, a hapten, enzyme fragment, co-factor or subunit, nanoparticle, radiolabel, contrast agent, and/or quantum dots.

In some embodiments, the released reporter molecule is detected by at least one of color detectors, turbidimeters, fluorometers, electrical signal detectors, lateral flow assays, electrophoresis, chromatography, or magnetic readers.

In other embodiments, the released reporter molecule includes a small molecule that is degradable upon cleavage of the reporter molecule to release a plurality of detectable molecules indicative of the cleavage.

In other embodiments, the reporter molecule is a co-factor that activates an enzyme to generate color, fluorescence, or breakdown of a substrate into a detectable molecule.

In other embodiments, the reporter molecule include a DNA sequence that is readily amplified.

In some embodiments, the Cas nuclease effector protein comprises a Cas9, Cas 12, or Cas 13 nuclease effector protein.

In some embodiments, the target-specific nucleic acid of the barcode sequence is configured to target one or multiple genes of an organism or multiple organisms.

In some embodiments, the plurality of barcode sequences include first barcode sequences that include first target-specific nucleic acids and first reporter molecules and second barcode sequences that include second target-specific nucleic acids and second reporter molecules that differ from the first target-specific nucleic acids and first reporter molecules.

In some embodiments, the Cas nuclease effector protein includes a first Cas nuclease effector protein complexed with first guide nucleic acid that is complementary or hybridizes to the first target specific nucleic acids or a first targeted nucleic acid and a second Cas nuclease effector protein complexed with a second guide nucleic acid that is complementary or hybridizes to the second target-specific nucleic acids or a second targeted nucleic acid.

In some embodiments, the plurality of barcode sequences include barcode sequence having differing target specific nucleic acids and reporter molecules and the Cas nuclease includes Cas nuclease with differing guide nucleic acids that are complementary or hybridize to the differing target-specific nucleic acids or differing targeted nucleic acids.

In some embodiments, the plurality of barcode sequences are directly or indirectly linked to a plurality of magnetic beads.

In some embodiments, the target-specific nucleic acid sequence of the barcode sequences includes a first end linked to the magnetic bead and a second terminal end linked to the reporter molecule.

In some embodiments, the plurality of barcode sequences are linked to the magnetic bead using a click reaction chemistry.

In some embodiments, the plurality of barcode sequences are topographically and/or spatially arranged on an outer surface of the magnetic beads.

In some embodiments, the method further includes magnetically separating the magnetic beads from released reporter molecules.

Other embodiments described herein relate to a CRISPR-based molecular beacon system that includes a CRISPR-Cas protein engineered with metallic nanoparticle acceptors and a tailed-guiding RNA (gRNA) terminally modified with donor fluorescent dye(s) that is configured to assemble in the presence of target DNA, forming a CRISPR complex with enhanced energy transfer and fluorescence quenching. The target DNA interacts with the tail of the gRNA molecules to form dsDNA that triggers its binding to CRISPR-Cas protein bringing the donor fluorescent dyes to the proximity of metal nanoparticle acceptors.

### BRIEF DESCRIPTION OF THE DRAWINGS

FIG. 1 is a schematic illustration of CRISPR based system.

FIG. 2 is a schematic illustration of CRISPR based system for molecular testing of nucleic acids.

FIG. 3 is a schematic illustrate of CRISPR based system for immunological testing of proteins.

FIG. 4 is a schematic illustration of CRISPR flares used with an assay.

FIG. 5 is a schematic illustration of the CRISPR beacons for the detection of target DNA. Each beacon system comprises two major components: (1) an inactive CRISPR-Cas9 that is specifically engineered with gold nanoparticle (AuNP), and (2) single-stranded guiding RNA (sgRNA) that is tailed with a target-specific DNA terminally modified with a fluorescent dye. The interaction of the target DNA with the sgRNA activates CRISPR-Cas9 to bind to the formed dsDNA bringing fluorescent dye close to AuNP, which results in quenching its fluorescence signal indicating the presence of a specific target sequence.

### DETAILED DESCRIPTION

To facilitate the understanding of this invention, a number of terms are defined below. Terms defined herein have meanings as commonly understood by a person of ordinary skill in the areas relevant to the present invention. Terms such as “a”, “an”, and “the” are not intended to refer to only a singular entity but also plural entities and also includes the general class of which a specific example may be used for illustration. The terminology herein is used to describe specific aspects of the invention, but their usage does not delimit the invention, except as outlined in the claims.

Throughout the description, where compositions are described as having, including, or comprising, specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the compositions and methods described herein remains operable. Moreover, two or more steps or actions can be conducted simultaneously.

As used herein, the term “about” or “approximately” refers to a quantity, level, value, number, frequency, percentage, dimension, size, amount, weight or length that varies by as much as 15%, 10%, 9%, 8%, 7%, 6%, 5%, 4%, 3%, 2% or 1% to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length ±15%, ±10%, ±9%, ±8%, ±7%, ±6%, ±5%, +4%, ±3%, ±2%, or ±1% about a reference quantity, level, value, number, frequency, percentage, dimension, size, amount, weight or length.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation. “Optional” or “optionally” means that the subsequently described circumstance may or may not occur, so that the description includes instances where the circumstance occurs and instances where it does not.

The term “sample” as used herein is used in its broadest sense and includes environmental and biological samples. Environmental samples include material from the environment, such as soil and water. Biological samples may be animal, including, human, fluid, e.g., blood, plasma, and serum; solid, e.g., stool; tissue; liquid foods, e.g., milk; and solid foods, e.g., vegetables. A biological sample may comprise a cell, tissue extract, body fluid, chromosomes or extrachromosomal elements isolated from a cell, genomic DNA (in solution or bound to a solid support such as for Southern blot analysis), RNA (in solution or bound to a solid support such as for Northern blot analysis), cDNA (in solution or bound to a solid support) and the like.

The terms “nucleic acid”, “nucleotide”, “polynucleotide,” and “oligonucleotide” are used interchangeably and refer to a deoxyribonucleotide or ribonucleotide polymer, in linear or circular conformation, and in either single-or double-stranded form. For the purposes of the present disclosure, these terms are not to be construed as limiting with respect to the length of a polymer. The terms can encompass known analogues of natural nucleotides, as well as nucleotides that are modified in the base, sugar and/or phosphate moieties (e.g., phosphorothioate backbones). In general, an analogue of a particular nucleotide has the same base-pairing specificity; i.e., an analogue of A will base-pair with T.

The terms “polypeptide,” “peptide” and “protein” are used interchangeably to refer to a polymer of amino acid residues. The term also applies to amino acid polymers in which one or more amino acids are chemical analogues or modified derivatives of a corresponding naturally-occurring amino acids.

“Binding” refers to a sequence-specific, non-covalent interaction between macromolecules (e.g., between a protein and a nucleic acid). Not all components of a binding interaction need be sequence-specific (e.g., contacts with phosphate residues in a DNA backbone), as long as the interaction as a whole is sequence-specific. Such interactions are generally characterized by a dissociation constant (K_{d}) of 10⁻⁶ M⁻¹ or lower. “Affinity” refers to the strength of binding: increased binding affinity being correlated with a lower K_{d}.

In general, “CRISPRs” (Clustered Regularly Interspaced Short Palindromic Repeats), also known as SPIDRs (SPacer Interspersed Direct Repeats), refer a family of DNA loci that are usually specific to a particular bacterial species.

“CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus. In some embodiments, one or more elements of a CRISPR system is derived from a class 1 type I or type III CRISPR system. In some embodiments, one or more elements of a CRISPR system is derived from a class 2 type II, or type V CRISPR system. In some embodiments, one or more elements of a CRISPR system is derived from a particular organism comprising an endogenous CRISPR system, such as Streptococcus pyogenes.In general, a CRISPR system is characterized by elements that promote the formation of a CRISPR complex at the site of a target sequence (also referred to as a protospacer in the context of an endogenous CRISPR system). In the context of formation of a CRISPR complex, “target sequence” refers to a sequence to which a guide sequence is designed to have complementarity, where hybridization between a target sequence and a guide sequence promotes the formation of a CRISPR complex. Full complementarity is not necessarily required, provided there is sufficient complementarity to cause hybridization and promote formation of a CRISPR complex. A target sequence may comprise any polynucleotide, such as DNA or RNA polynucleotides.

In the methods of the disclosure, one or more targeted nucleases as described herein cleave (e.g., create one or more single-stranded nicks and/or one or more double-stranded breaks (DSBs)) in the target sequence at a predetermined site.

“Genetically modified” refers to a modification made to a nucleic acid such that the sequence of the nucleic acid is altered in comparison to the nucleic acid prior to being modified. Genetically modifying a cell refers to modifying cellular nucleic acid within a cell, including genetic modifications to endogenous and/or exogenous nucleic acids within the cell. Genetic modifications can comprise deletions, insertions, integrations of exogenous DNA, gene correction and/or gene mutation.

“Cleavage” refers to the breakage of the covalent backbone of a DNA molecule. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. DNA cleavage can result in the production of either blunt ends or staggered ends. In certain embodiments, fusion polypeptides are used for targeted double-stranded DNA cleavage.

The term “sequence” refers to a nucleotide sequence of any length, which can be DNA or RNA; can be linear, circular or branched and can be either single-stranded or double stranded. The term “donor sequence” refers to a nucleotide sequence that is inserted into a genome. A donor sequence can be of any length, for example between 2 and 100,000,000 nucleotides in length (or any integer value therebetween or there above), preferably between about 100 and 100,000 nucleotides in length (or any integer therebetween), more preferably between about 2000 and 20,000 nucleotides in length (or any value therebetween) and even more preferable, between about 5 and 15 kb (or any value therebetween).

A “target site” or “target sequence” is a nucleic acid sequence that defines a portion of a nucleic acid to which a binding molecule will bind, provided sufficient conditions for binding exist.

Embodiments described herein relate to the use of CRISPR based technologies in a system for detecting an analyte in a sample, a diagnostic device of the analyte detection system, use of the analyte detection system as a diagnostic agent, a kit-of-parts for detecting an analyte, such as nucleic acids, a method for detecting an analyte, and a method for diagnosing a disease state of a subject. The detection system comprises a CRISPR-Cas system which includes an effector protein and one or more guide RNAs having a guide sequence. The guide sequence can be capable of targeting the effector protein to a target sequence of a target, such as a target nucleic acid, and the effector protein can exhibit target-activated nucleic acid cleavage activity capable of cleaving reporter molecules that is indicative of the presence of the analyte in a sample.

In some embodiments, the system for detecting one or more analytes in a sample includes a plurality of magnetic beads, a plurality of analyte capture molecules extending from the surfaces of the beads, a CRISPR Cas nuclease effector protein and a plurality of barcode sequences that are configured to specifically and selectively bind to the one or more analyte in the sample. Each barcode sequence includes a nucleic acid and a reporter molecule. The Cas nuclease effector protein is configured to cleave the nucleic acid of the barcode sequence and release the reporter molecule after binding of the barcode sequence to the analyte in the sample. The released reporter molecule can then be detected to indicate the presence and/or quantity of the analyte in the sample.

In some embodiments, the analyte is a targeted nucleic acid in the sample and the analyte capture molecule includes and/or defines the barcode sequence. The barcode sequence can include a single-stranded target-specific nucleic acid sequence that is complementary to or hybridizes with at least a portion of a targeted nucleic acid.

The target-specific nucleic acid sequence can be of any desired length. For example, the target-specific nucleic acid sequence can be about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, the target-specific nucleic acid sequence is less than about 75, 50, 45, 40, 35, 30, 25, 20, 15, 12, or fewer nucleotides in length.

The barcode sequence can be immobilized on a surface of the magnetic bead by covalently or non-covalently linking to the surface of the magnetic bead. For immobilizing on or linking to a surface of the magnetic bead, the barcode sequence can comprise a functional group for immobilization. It is noted that the functional group for immobilization can be located anywhere in the barcode sequence. For example, the functional group for immobilization can be at the 5′-end of barcode sequence. Alternatively, the functional group for immobilization can be at the 3′-end of the barcode sequence.

The functional group can include any functional group that can react with another molecule or functional group and form a covalent or non-covalent linkage. Exemplary functional groups include, but are not limited to, acetal, acetylene, acid amide, acid anhydride, acid imide, alcohol, aldehyde, allene, amidine, amine or amino, aminooxy, azanol, azide, azo-compound, azoxy compound, carbamate, carbodiimides, carboxylic acid, cyanate, cyanide, diazo, diazol, disulfide, enamine, epoxy, ester, ether, halide, hydrazide, hydrazine, hydrazone, hydroxamic acid, hydroxyl, imide ester, imines, isocyanate, isonitrile, isothiocyanate, ketal, ketone, mercaptan, nitrile, nitro, nitrone, nitroso, ortho esters, oxide, oxime, phenol, phosphate group, pseudo-urea, semicarbazide, sulfenic acid, sulfide, sulfinic acid, sulfite, sulfone, sulfonic acid, sulfoxide, sulfuric ester, sulfur hydroxamic acid, thiocyanate, thiol, and urea.

In some embodiments, the functional group can be one member of a binding pair. A “binding pair”, “coupling molecule pair” and “coupling pair” are used interchangeably and without limitation herein to refer to the first and second molecules or functional groups that specifically bind to each other. For example, the binding can be through one or more of a covalent bond, a hydrogen bond, an ionic bond, and a dative bond. In some embodiments one member of the binding pair is conjugated with a solid substrate while the second member is conjugated with the linker. A binding pair can be used for linking the linker to the substrate, and/or for linking the linker to the analyte-related molecule.

Exemplary coupling molecule pairs also include, without limitations, any haptenic or antigenic compound in combination with a corresponding antibody or binding portion or fragment thereof (e.g., digoxigenin and anti-digoxigenin; mouse immunoglobulin and goat anti-mouse immunoglobulin) and non-immunological binding pairs (e.g., biotin-avidin, biotin-streptavidin), hormone (e.g., thyroxine and cortisol-hormone binding protein), receptor-receptor agonist, receptor-receptor antagonist (e.g., acetylcholine receptor-acetylcholine or an analog thereof), IgG-protein A, lectin-carbohydrate, enzyme-enzyme cofactor, enzyme-enzyme inhibitor, and complementary oligonucleotide pairs capable of forming nucleic acid duplexes). The coupling molecule pair can also include a first molecule that is negatively charged and a second molecule that is positively charged.

One example of using coupling pair conjugation is the biotin-avidin or biotin-streptavidin conjugation. In this approach, one of the members of the coupling pair is biotinylated and the other is conjugated with avidin or streptavidin. Many commercial kits are also available for biotinylating molecules. For example, an aminooxy-biotin (AOB) can be used to covalently attach biotin to a molecule with an aldehyde or ketone group. In some embodiments, the functional group is biotin or a variant thereof.

Other examples for forming a coupling pair include click chemistry. As used herein “click chemistry” refers to a class of small molecule reactions which can be used for the linking of a binding pair and is not a single specific reaction but rather describes the method of generating products by mimicking nature which produces substance by joining of small modular units. Although useful for biochemical reactions, click chemistry is not limited to biological conditions. Click reactions are efficient and easy to used, occurring in one pot without any special precautions against water and air, do not produce offensive (e.g., not toxic) byproducts, and, because they are characterized by a high thermodynamic driving force that drives the reaction quickly to a single reaction product, require minimal or no final isolation and purification. Examples of click chemistry includes the copper-catalyzed reaction of an azide with an alkyne to form a 5-membered heteroatom ring (e.g., a Cu(I)-catalyzed azide-alkyne cycloaddition), the thiol-Michael Addition reaction such as reaction of a thiol group with a maleimide group, strain-promoted azide-alkyne cycloaddition, strain-promoted alkyne-nitrone cycloaddition, reactions of strained alkenes, alkene and azide [3+2] cycloaddition, alkene and tetrazine inverse-demand Diels-Alder, and alkene and tetrazole photoclick reaction.

The magnet beads to which analyte capture molecule or barcode sequence are bound can include a magnetic bead support having a diameter of about 1 μ to about 10 mm comprising a surface and a core. The surface can include one or more surface modifications, such as thiol groups, streptavidin, or biotin, for binding the analyte capture molecules or barcode sequences. The magnetic core may comprise one or more magnetic or magnetizable materials including, for example, iron, an iron oxide, cobalt a cobalt oxide, nickel, a nickel oxide, or combinations thereof.

In some embodiments, the plurality of barcode sequences are directly or indirectly linked to a plurality of plurality of magnetic beads. The plurality of barcode sequences can be topographically and/or spatially arranged on an outer surface of the magnetic beads.

In some embodiments, the plurality of barcode sequences can be linked to the magnetic beads using a click reaction chemistry. By way of example, differing DNA barcodes terminally modified with maleimide groups at the 5′ end can be used to prepare a mixed DNA corona on the surface of thiolated magnetic beads.

In some embodiments, the target-specific nucleic acid sequences of the barcode sequences can each include a first end linked to the magnetic bead and a second terminal end linked to the reporter molecule. The reporter molecule is configured to be cleaved from the barcode sequence by a Cas nuclease effector protein after binding of target-specific nucleic acid to the at least portion of the targeted nucleic acid.

In some embodiments, the reporter molecule includes at least one of a dye including a fluorescent or a colorimetric dye, a marker molecule or polymer having a defined molecular weight, an electrically active or detectable marker, a hapten, enzyme fragment, co-factor or subunit, nanoparticle, radiolabel, contrast agent, quantum dots, and/or any other agent that is detectable by, for example, color detectors, turbidimeters, fluorometers, electrical signal detectors, lateral flow assays, electrophoresis, chromatography, and magnetic readers.

In other embodiments, the reporter molecule includes a small molecule that is degradable upon cleavage of the reporter molecule to release a plurality of detectable molecules indicative of the cleavage.

In other embodiments, the reporter molecule is a co-factor that activates an enzyme to generate color, fluorescence, or breakdown of a substrate into a detectable molecule.

In some embodiments, the reporter molecule is linked to the target-specific nucleic acid of the barcode sequence with a functional group as described herein.

In some embodiments, the Cas nuclease forms a CRISPR complex with a guide RNA sequence that includes a nucleotide sequence that hybridizes to or is complementary to the target-specific nucleic acid sequence or the portion of the targeted nucleic acid. That is, the guide nucleic acid has a guide sequence which is capable of targeting an effector protein to a target sequence of the targeted nucleic acid or the barcode sequence.

Typically, the CAS effector proteins can belong to class 2 CRISPR-Cas systems. Class 2 Cas effector protein can include type II Cas9 and Cas9-like proteins, type V Cas12 and Cas 12-like proteins, such as subtype V-A Cas12 (Cpf1, or Cas12a), subtype V-B Cas12 (Cas12b, or C2c1) and subtype V-C Cas12 (C2c3), and type VI Cas13 and Cas13-like proteins, such as Cas 13a (C2c2) and Cas13b (C2c6).

In some embodiments, the Cas endonuclease effector protein can include a CAS endonuclease protein of a class 2 CRISPR-Cas system. In some embodiments, the CRISPR-Cas system is a type II, V or VI system. In a type II system, the effector protein is Cas9 or an effector protein having similar cleavage activity as Cas9. In case of a class 2 system, the effector protein may be Cas12 or an effector protein having similar cleavage activity as Cas12. The Cas12 or Cas12-like effector protein may, for example, be selected from Cas12a, Cas12b, Cas12c, Cas12d and Cas12e.

The Cas endonuclease effector protein may comprise a Cas endonuclease effector protein from more than one CRISPR-Cas system, for example, wherein the more than one CRISPR-Cas systems are different.

The programmability and specificity of the RNA-guided class 2 Cas effector proteins, such as Cpf1, make them suitable switchable nucleases for specific cleavage of nucleic acids. The class 2 Cas effector proteins, such as Cpf1, may be engineered to provide and take advantage of improved collateral non-specific cleavage of DNA, preferably ssDNA. Accordingly, engineered class 2 Cas effector proteins, such as Cpf1, may provide suitable platforms for nucleic acid detection.

An Cpf1 effector protein may be from an organism from a genus comprising Streptococcus, Campylobacter, Nitratifactor, Staphylococcus, Parvibaculum, Roseburia, Neisseria, Gluconacetobacter, Azospirillum, Sphaerochaeta, Lactobacillus, Eubacterium, Corynebacter, Carnobacterium, Rhodobacter, Listeria, Paludibacter, Clostridium, Lachnospiraceae, Clostridiaridium, Leptotrichia, Francisella, Legionella, Alicyclobacillus, Methanomethyophilus, Porphyromonas, Prevotella, Bacteroidetes, Helcococcus, Letospira, Desulfovibrio, Desulfonatronum, Opitutaceae, Tuberibacillus, Bacillus, Brevibacilus, Methylobacterium or Acidaminococcus.

In some embodiments, the effector protein can be Cas 9, Cas12a, Cas13a, CasX, CasPhi or Cas14. In some embodiments, the effector protein is Cas12a, also known as Cpf1.

The “guide nucleic acid,” “guide sequence,” “crRNA,” “guide RNA,” or “single guide RNA,” or “gRNA” refers to a polynucleotide comprising any polynucleotide sequence having sufficient complementarity with or that hybridizes to the target-specific nucleic acid sequence or the portion of the targeted nucleic acid sequence and to direct sequence-specific binding of a CRISPR complex comprising the guide sequence and a Cas effector protein to the target-specific nucleic acid sequence or the portion of the targeted nucleic acid sequence.

In some embodiments, the degree of complementarity, when optimally aligned using a suitable alignment algorithm, is about or more than about 50%, 60%, 75%, 80%, 85%, 90%, 95%, 97.5%, 99%), or more. Optimal alignment can be determined with the use of any suitable algorithm for aligning sequences. Exemplary algorithms for determining optimal alignment include, but are not limited to, the Smith-Waterman algorithm, the Needleman-Wunsch algorithm, algorithms based on the Burrows-Wheeler Transform, ClustalW, Clustal X, BLAT, Novoalign (Novocraft Technologies; available at www.novocraft.com), ELAND (Illumina, San Diego, Calif.), SOAP (available at soap.genomics.org.cn), and Maq (available at maq.sourceforge.net).

The guide nucleic acid sequence can be any length. For example, the guide nucleic acid strand can be about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, the guide nucleic acid sequence is less than about 75, 50, 45, 40, 35, 30, 25, 20, 15, 12, or fewer nucleotides in length. Preferably, the guide nucleic acid sequence is 10-30 nucleotides long.

In some embodiments, the plurality of barcode sequences include first barcode sequences that include first target-specific nucleic acids and first reporter molecules and second barcode sequences that include second target-specific nucleic acids and second reporter molecules that differ from the first target-specific nucleic acids and first reporter molecules so as to detect different analytes and/or nucleic acids in the sample.

In other embodiments, the Cas nuclease includes a first Cas nuclease complexed with first guide RNA that is complementary or hybridizes to the first target specific nucleic acids or a first targeted nucleic acid and a second Cas nuclease complexed with a second guide RNA that is complementary or hybridizes to the second target-specific nucleic acids or a second targeted nucleic acid so as to detect different analytes and/or nucleic acids in the sample.

In still other embodiments, the plurality of barcode sequences include barcode sequence having differing target specific nucleic acids and reporter molecules and the Cas nuclease includes Cas nuclease with differing guide RNAs that are complementary or hybridize to the differing target-specific nucleic acids or differing targeted nucleic acids so as to detect different analytes and/or nucleic acids in the sample.

In some embodiments, the target-specific nucleic acid of the barcode sequence can be configured to target a nucleic sequence, such as one or multiple genes of an organism or multiple organisms. The target sequence can comprise any polynucleotide, such as DNA or RNA polynucleotides. In some embodiments, a target sequence is located in the nucleus or cytoplasm of a cell, and can include nucleic acids in or from mitochondrial, organelles, vesicles, liposomes or particles present within the cell. The target sequence can be any desired nucleic acid. Further, the target sequence can be naturally occurring or synthetic nucleic acid. Thus, in some embodiments, the target sequence is a naturally occurring nucleic acid. A naturally occurring sequence includes a nucleic acid isolated and/or purified from a natural source.

The target sequence can be within a double-stranded or single-stranded region of the target. In some embodiments, the target sequence can be a sequence within a DNA molecule. The target DNA molecule can be genomic DNA, cell free DNA (cfDNA), mitochondrial DNA, cDNA or the like. In some embodiments, the target sequence can be a sequence within an RNA molecule. The RNA molecule can be messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (IncRNA), or small cytoplasmic RNA (scRNA). It is noted that the target nucleic acid can be a naturally occurring nucleic acid or a synthetic nucleic acid.

In some embodiments described herein, the target sequence is from an organism, including but not limited to a prokaryote, eukaryote, archaeabacteria, animal, plant, protist, parasite, fungus, or bacterium. In some embodiments described herein, the target sequence is from a virus. In some embodiments described herein, the target sequence is from a human. In some embodiments described herein, the target sequence is from a pathogenic organism. In some embodiments described herein, the target sequence is from a non-pathogenic organism.

In some embodiments described herein, the target sequence is from a bacterium, which can be a pathogenic or non-pathogenic bacterial species. Non-limiting examples of pathogenic bacteria that can comprise the target sequence include spirochetes (e.g. Borrelia), actinomycetes (e.g. Actinomyces), mycoplasmas, Rickettsias, Gram negative aerobic rods, Gram negative aerobic cocci, Gram negatively facultatively anaerobic rods (e.g. Erwinia and Yersinia), Gram-negative cocci, Gram negative coccobacilli, Gram positive cocci (e.g. Staphylococcus and Streptococcus), endospore-forming rods, and endospore-forming cocci.

Non-limiting examples of bacterial pathogens include Bacillus, Brucella, Burkholderia, Francisella, Yersinia, Streptococcus, Haemophilus, Nisseria, Listeria, Clostridium, Klebsiella, Legionella, Escherichia (e.g., E. coli), Mycobacterium, Staphylococcus, Campylobacter, Vibrio, and Salmonella, as well as drug and multidrug resistant strains and highly virulent strains of these pathogenic bacteria. Non-limiting examples of known food-borne bacterial pathogens include Salmonella, Clostridium, Campylobacter spp., Staphylococcus, Salmonella, Escherichia (e.g., E. coli), and Listeria.In some embodiments, non-limiting examples of bacterial pathogens include Bacillus anthracis, Brucella abortus, Brucella melitensis, Brucella suis, Burkholderia mallei, Burkholderia pseudomallei, Francisella tularensis, Yersinia pestis, Streptococcus Group A and B, MRSA, Streptococcus pneumonia, Haemophilus influenza, Nisseria meningitides, Listeria monocytegenes, Clostridium difficile, Klebsiella, highly virulent pathogenic strains of E. coli, Mycobacterium tuberculosis, Staphylococcus aureus, Campylobacter spp, Salmonella spp, and Clostridium perfringens, as well as drug and multidrug resistant strains and highly virulent strains of these pathogenic bacteria. In some embodiments, non-limiting examples of known food-borne bacterial pathogens include Salmonella, non typhoidal Clostridium perfringens, Campylobacter spp., Staphylococcus aureus, Salmonella, nontyphoidal, Campylobacter spp., E. coli (STEC) 0157, and Listeria monocytogenese. In some embodiments of the various aspects described herein, the target sequence is from a Borrelia bacterial species, such as Borrelia burgdorferi.

In some embodiments described herein, the target sequence is from a fungus, which can be a pathogenic or non-pathogenic fungal species. Non-limiting examples of fungi that can comprise the target sequence include yeast and molds, such as Aspergillus, Cladosporium, Epicoccum, Penicillium, Acremonium, Exophiala, Phialophora, Trichoderma, Fusarium, Phoma, Mucorales, Geotrichum, Candida, and Claviceps.

In some embodiments of the various aspects described herein, the target nucleic acid is a viral DNA or RNA. For example, the target nucleic acid is from an RNA virus.

In some embodiments, the RNA virus is Group III (i.e., double stranded RNA (dsRNA)) virus. In some embodiments of the various aspects described herein, the Group III RNA virus belongs to a viral family selected from the group consisting of: Amalgaviridae, Birnaviridae, Chrysoviridae, Cystoviridae, Endornaviridae, Hypoviridae, Megabirnaviridae, Partitiviridae, Picobirnaviridae, Reoviridae (e.g., Rotavirus), Totiviridae, Quadriviridae. In some embodiments of the various aspects described herein, the Group III RNA virus belongs to the Genus Botybirnavirus. In some embodiments of the various aspects described herein, the Group III RNA virus is an unassigned species selected from the group consisting of: Botrytis porri RNA virus 1, Circulifer tenellus virus 1, Colletotrichum camelliae filamentous virus 1, Cucurbit yellows associated virus, Sclerotinia sclerotiorum debilitation-associated virus, and Spissistilus festinus virus 1.

In some embodiments described herein, the RNA virus is a Group IV (i.e., positive-sense single stranded (ssRNA)) virus. In some embodiments of the various aspects described herein, the Group IV RNA virus belongs to a viral order selected from the group consisting of: Nidovirales, Picornavirales, and Tymovirales. In some embodiments described herein, the Group IV RNA virus belongs to a viral family selected from the group consisting of: Arteriviridae, Coronaviridae (e.g., Coronavirus, SARS-COV), Mesoniviridae, Roniviridae, Dicistroviridae, Iflaviridae, Marnaviridae, Picornaviridae (e.g., Poliovirus, Rhinovirus (a common cold virus), Hepatitis A virus), Secoviridae (e.g., sub Comovirinae), Alphaflexiviridae, Betaflexiviridae, Gammaflexiviridae, Tymoviridae, Alphatetraviridae, Alvernaviridae, Astroviridae, Barnaviridae, Benyviridae, Bromoviridae, Caliciviridae (e.g., Norwalk virus), Carmotetraviridae, Closteroviridae, Flaviviridae (e.g., Yellow fever virus, West Nile virus, Hepatitis C virus, Dengue fever virus, Zika virus), Fusariviridae, Hepeviridae, Hypoviridae, Leviviridae, Luteoviridae (e.g., Barley yellow dwarf virus), Polycipiviridae, Narnaviridae, Nodaviridae, Permutotetraviridae, Potyviridae, Sarthroviridae, Statovirus, Togaviridae (e.g., Rubella virus, Ross River virus, Sindbis virus, Chikungunya virus), Tombusviridae, and Virgaviridae. In some embodiments of the various aspects described herein, the Group IV RNA virus belongs to a viral genus selected from the group consisting of: Bacillariornavirus, Dicipivirus, Labyrnavirus, Sequiviridae, Blunervirus, Cilevirus, Higrevirus, Idaeovirus, Negevirus, Ourmiavirus, Polemovirus, Sinaivirus, and Sobemovirus. In some embodiments of the various aspects described herein, the Group IV RNA virus is an unassigned species selected from the group consisting of: Acyrthosiphon pisum virus, Bastrovirus, Blackford virus, Blueberry necrotic ring blotch virus, Cadicistrovirus, Chara australis virus, Extra small virus, Goji berry chlorosis virus, Hepelivirus, Jingmen tick virus, Le Blanc virus, Nedicistrovirus, Nesidiocoris tenuis virus 1, Niflavirus, Nylanderia fulva virus 1, Orsay virus, Osedax japonicus RNA virus 1, Picalivirus, Plasmopara halstedii virus, Rosellinia necatrix fusarivirus 1, Santeuil virus, Secalivirus, Solenopsis invicta virus 3, Wuhan large pig roundworm virus. In some embodiments of the various aspects described herein, the Group IV RNA virus is a satellite virus selected from the group consisting of: Family Sarthroviridae, Genus Albetovirus, Genus Aumaivirus, Genus Papanivirus, Genus Virtovirus, and Chronic bee paralysis virus.

In some embodiments described herein, the RNA virus is a Group V (i.e., negative-sense ssRNA) virus. In some embodiments of the various aspects described herein, the Group V RNA virus belongs to a viral phylum or subphylum selected from the group consisting of: Negarnaviricota, Haploviricotina, and Polyploviricotina. In some embodiments described herein, the Group V RNA virus belongs to a viral class selected from the group consisting of: Chunqiuviricetes, Ellioviricetes, Insthoviricetes, Milneviricetes, Monjiviricetes, and Yunchangviricetes. In some embodiments described herein, the Group V RNA virus belongs to a viral order selected from the group consisting of: Articulavirales, Bunyavirales, Goujianvirales, Jingchuvirales, Mononegavirales, Muvirales, and Serpentovirales. In some embodiments of the various aspects described herein, the Group V RNA virus belongs to a viral family selected from the group consisting of: Amnoonviridae (e.g., Taastrup virus), Arenaviridae (e.g., Lassa virus), Aspiviridae, Bornaviridae (e.g., Borna disease virus), Chuviridae, Cruliviridae, Feraviridae, Filoviridae (e.g., Ebola virus, Marburg virus), Fimoviridae, Hantaviridae, Jonviridae, Mymonaviridae, Nairoviridae, Nyamiviridae, Orthomyxoviridae (e.g., Influenza viruses), Paramyxoviridae (e.g., Measles virus, Mumps virus, Nipah virus, Hendra virus, and NDV), Peribunyaviridae, Phasmaviridae, Phenuiviridae, Pneumoviridae (e.g., RSV and Metapneumovirus), Qinviridae, Rhabdoviridae (e.g., Rabies virus), Sunviridae, Tospoviridae, and Yueviridae. In some embodiments described herein, the Group V RNA virus belongs to a viral genus selected from the group consisting of: Anphevirus, Arlivirus, Chengtivirus, Crustavirus, Tilapineviridae, Wastrivirus, and Deltavirus (e.g., Hepatitis D virus).

In some embodiments described herein, the RNA virus is a Group VI RNA virus, which comprise a virally encoded reverse transcriptase. In some embodiments of the various aspects described herein, the Group VI RNA virus belongs to the viral order Ortervirales. In some embodiments of the various aspects described herein, the Group VI RNA virus belongs to a viral family or subfamily selected from the group consisting of: Belpaoviridae, Caulimoviridae, Metaviridae, Pseudoviridae, Retroviridae (e.g., Retroviruses, e.g. HIV), Orthoretrovirinae, and Spumaretrovirinae. In some embodiments described herein, the Group VI RNA virus belongs to a viral genus selected from the group consisting of: Alpharetrovirus (e.g., Avian leukosis virus; Rous sarcoma virus), Betaretrovirus (e.g., Mouse mammary tumour virus), Bovispumavirus (e.g., Bovine foamy virus), Deltaretrovirus (e.g., Bovine leukemia virus; Human T-lymphotropic virus), Epsilonretrovirus (e.g., Walleye dermal sarcoma virus), Equispumavirus (e.g., Equine foamy virus), Felispumavirus (e.g., Feline foamy virus), Gammaretrovirus (e.g., Murine leukemia virus; Feline leukemia virus), Lentivirus (e.g., Human immunodeficiency virus 1; Simian immunodeficiency virus; Feline immunodeficiency virus), Prosimiispumavirus (e.g., Brown greater galago prosimian foamy virus), and Simiispumavirus (e.g., Eastern chimpanzee simian foamy virus).

In some embodiments described herein, the RNA virus is selected from influenza virus, human immunodeficiency virus (HIV), severe acute respiratory syndrome coronavirus 2(SARS-CoV-2 ), and SARS-associated coronavirus (SARS-CoV). In some embodiments of the various aspects described herein, the RNA virus is influenza virus. In some embodiments of the various aspects described herein, the RNA virus is immunodeficiency virus (HIV). In some embodiments described herein, the RNA virus is severe acute respiratory syndrome coronavirus 2 (SARS-COV-2). In some embodiments described herein, the RNA virus is SARS-associated coronavirus (SARS-COV). In some embodiments described herein, the RNA virus is any known RNA virus.

In some embodiments described herein, the viral RNA is an RNA produced by a virus with a DNA genome, i.e., a DNA virus. As a non-limiting example the DNA virus is a Group I (dsDNA) virus, a Group II (ssDNA) virus, or a Group VII (dsDNA-RT) virus. In some embodiments of the various aspects described herein, the RNA produced by a DNA virus comprises an RNA transcript of the DNA genome.

The target nucleic acids as described herein may be obtained from a biological sample or an environmental sample. The biological sample or environmental sample may originate from a subject as described herein. The biological sample may be obtained from blood, plasma, serum, urine, stool, sputum, mucous, lymph fluid, synovial fluid, bile, ascites, pleural effusion, seroma, saliva, cerebrospinal fluid, aqueous or vitreous humour, or any bodily secretion, a transudate, an exudate, such as fluid obtained from an abscess or any other site of infection or inflammation), or fluid obtained from a joint, such as a normal joint or a joint effected by disease, such as rheumatoid arthritis, osteoarthritis, gout or septic arthritis, a swab of skin or mucosal membrane surface, or a combination thereof. Preferably, the biological sample may be obtained from blood, plasma, serum, urine, stool, sputum, mucous, saliva, or any bodily secretion, a transudate, an exudate, such as fluid obtained from an abscess or any other site of infection or inflammation), a swab of skin or mucosal membrane surface, or a combination thereof. The environmental sample may be obtained from food (e.g., fruit, vegetables, meat, beverage, etc.), paper surface, fabric, metal surface, wood or wood surface, plastic surface, soil, water, such as fresh water or waste water, saline water, atmospheric air or other gas sample, or a combination thereof.

It will be appreciated that the analyte to be detected need not be limited to a nucleic acid but can also include polypeptide. In embodiments where the analyte to be detected is a polypeptide, the plurality of barcode sequences can include a nucleic acid linked to antibody or antigen binding fragment thereof that can specifically bind to the polypeptide in the sample. For example, the barcode sequence can include a nucleic sequence with a first end and a second end. The first end can be linked to the antibody or antigen binding fragment thereof that specifically binds to the polypeptide and the second end includes a reporter molecule. Instead of binding the barcode sequence beads, analyte capture molecules that include a plurality of second antibody or antigen binding fragments thereof can be directly or indirectly linked to the magnetic beads. The second antibody or antigen binding fragments thereof specifically can bind the polypeptide. The barcode first antibody or antigen binding fragment of the barcode sequence can bind to polypeptides bound to the second antibody or antigen binding fragment immobilized on the surface of magnetic beads, and the nucleic acid of the barcode sequence can be cleaved from the barcode sequence by the Cas nuclease after binding of barcode sequence to the polypeptide to release the reporter molecule. The presence or level of the reporter molecule can be detected or measured to detect the presence or level of the polypeptide in the sample.

In some embodiments, the plurality of second antibodies or antigen binding fragments thereof can be topographically and/or spatially arranged on an outer surface of the magnetic beads.

In some embodiments, the second antibody or antigen binding fragment thereof binds to a different epitope of the polypeptide than the first antibody or antigen binding fragment thereof of the barcode sequence.

Other embodiments described herein relate to a method of detecting an analyte in a sample. The method includes providing a plurality of barcode sequences that are configured to specifically bind to the analyte in the sample. Each barcode sequence can include a nucleic acid and reporter molecule. The nucleic acid of the barcode sequence is configured to be cleaved from the barcode sequence by a Cas nuclease effector protein after binding of barcode sequence to the analyte. The barcode sequences are combined with the sample such that the barcode binds to the analyte in the sample. The Cas nuclease effector protein can be added to the combined barcode sequences and sample. The Cas nuclease effector protein cleaves the barcode sequences hybridized to the analyte to release the reporter molecule and presence or quantity of the released reporter molecule can be detected to indicate the presence or quantity of the analyte in the sample.

In some embodiments, the Cas nuclease effector protein added to the combined barcode sequences and sample can be incubated for a period time. Incubation time is sufficient to allow the guide nucleic acid sequence to hybridize with the target nucleic acid sequence or barcode sequence and form a CRISPR complex comprising the guide sequence, the target nucleic acid, barcode sequence, and the Cas effector protein. Incubation time can be 120 minutes or less.

For example, incubation time can be 2 hours, 1 hour, 30 minutes, 25 minutes, 20 minutes, 15 minutes, 10 minutes, 5 minutes, 2.5 minutes, 1 minute or less. In some embodiments, incubation time can be 15 minutes, 30 minutes, 60 minutes, 1.5 hours, 2 hours, 3 hours, or more. In some embodiments, the incubation time can be 1 minute or longer. As a non-limiting example, the incubation time can be at least 1 minute, at least 2 minutes, at least 3 minutes, at least 4 minutes, at least 5 minutes, at least 10 minutes, at least 20 minutes, at least 30 minutes, at least 40 minutes, at least 50 minutes, at least 60 minutes, at least 70 minutes, at least 80 minutes, at at least 90 minutes, at least 100 minutes, at least 110 minutes, or at least 120 minutes.

After a sufficient period of time to allow the Cas effector protein to cleave the barcode sequence on the magnetic beads to release the reporter molecule, the magnetic beads can be magnetically separating from released reporter moleculesby application of a magnetic field to the magnetic beads, which can be provided on a substrate or in a contain container. A magnetic field may be applied by forming a magnetic field at or near a surface or container containing the magnetic beads, or by bringing a surface or container containing the magnetic beads into the effective range of an existing magnetic field, for example, by moving the surface or container near the existing field and/or by reshaping a field to remove the magnetic beads.

The released reporter molecule separated from barcode sequence and magnetic beads can be detected to detect the presence of the analyte in the sample. The detection of the released reporter molecule may be performed according to any suitable assay format depending on the reporter molecule. Such assays of the reporter molecule can include western blots, radioimmunoassay like RIA (radio-linked immunoassay), ELISA (enzyme linked immunosorbent assay), “sandwich” immunoassays, immunoradiometric assays, fluorescent assays, chemiluminescence assays and electrochemiluminescence assays or suitable derivatives thereof.

In some embodiments, the reporter molecule includes at least one of a dye including a fluorescent a colorimetric dye, a marker molecule or polymer having a defined molecular weight, an electrically active or detectable marker, a hapten, enzyme fragment, co-factor or subunit, nanoparticle, radiolabel, contrast agent, and/or quantum dots.

In some embodiments, the released reporter molecule is detected by at least one of color detectors, turbidimeters, fluorometers, electrical signal detectors, lateral flow assays, electrophoresis, chromatography, or magnetic readers.

Details and further features of such assays would be known to the skilled person or can be derived from suitable literature sources such as the ebook Assay Guidance Manual, edited by G. Sitta Sittampalam, Bethesda (Md.): Eli Lilly & Company and the National Center for Advancing Translational Sciences; 2004.

The detection according may be a qualitative detection, a semi-quantitative detection or a quantitative detection. The term “qualitative detection” as used herein means that the method invention is capable of indicating whether a specific target analyte is present or not. The term “semi-quantitative detection” as used herein means that the method is capable of indicating whether a specific target analyte is present above a certain threshold with respect to its numbers or amount or concentration in a solution. The threshold may be suitably defined as would be known to the skilled person. The term “quantitative detection” as used herein means that the method is capable of indicating the approximate or exact numbers, amount or concentration of a specific target analyte used for the method. For the quantitative detection suitable control and/or calibration steps are required. Typically, the quantitative detection involves the interpretation of signals in comparison to a standard curve (e.g., a serial dilution of a known, purified target RNA polynucleotide) in order to precisely calculate the concentrations of target analyte in various samples.

In some embodiments, the quantitative detection can be performed by averaging the triplicate of the standards readings and by deducting the reading of the blank control sample. Subsequently, a standard curve is plotted and the line of best fit is identified so that the concentration of the samples can be determined. Any dilutions made need to be adjusted for at this stage. Alternatively, the signal data may be plotted using semi-log, log/log, log/logit or derivatives thereof in 4 or 5 parameter logistic models. Using software based/automated solutions suitable graphing approaches may be implemented. The approach further envisages the use of linear regression, e.g. within a software package, which allows for additional control possibilities. Further details would be known to the skilled person or can be derived from suitable literature sources.

The system described herein may be used in a medical device. Preferably, the nucleic acid detection system for use in medical applications for detection of nucleic acid.

The term “medical applications” as used herein is meant to include, for example, methods for diagnosing a disease state of a subject. The term “subject” as used herein is meant to include the human and animal body and plants, and the terms “individual” and “patient”. The terms “human” and “nonhuman” as used herein, are meant to include all animals, such as mammals, including humans. The term “individual” as used herein is meant to include any human or nonhuman entity. Humans and/or non-humans, such as domestic animals (i.e., pets, livestock, zoo animals, equines, etc.), may be subjected to the medical applications.

The system as described herein can be used as a diagnostic agent. The diagnostic agent may be used to diagnose a disease state of a subject as described herein.

There is further provided a system as described herein for use in detecting in vitro, in vivo or ex vivo pathogenic nucleic acids, wherein the pathogenic nucleic acids are pathogenic DNA. Preferably, the nucleic acid detection system is a nucleic acid detection system.

The nucleic acid detection system as described herein can be embodied on devices, in particular diagnostic devices. The device may be capable of defining multiple individual discrete volumes within the device, or a single individual discrete volume. As used herein an “individual discrete volume” refers to a discrete space, such as a container, receptacle, or other defined volume or space that can be defined by properties that prevent and/or inhibit migration of target molecules, for example a volume or space defined by physical properties such as walls a well or tube, which may be impermeable or semipermeable, or as defined by other means such as chemical, diffusion rate limited, electro-magnetic, or light illumination, or any combination thereof that can contain a sample within a defined space. The individual discrete volume may typically include a fluid medium (e.g., an aqueous solution, an oil, a buffer, etc.). Exemplary discrete volumes or spaces useful in the disclosed methods include tubes (e.g., centrifuge tubes, micro-centrifuge tubes, test tubes, cuvettes, and conical tubes), bottles (e.g., glass bottles, plastic bottles, ceramic bottles, Erlenmeyer flasks, and scintillation vials), wells (such as wells in a plate), plates, pipettes, and pipette tips.

Samples comprising target nucleic acids may be exposed to one or more of the discrete volumes each comprising a guide nucleic acid, barcode sequence, magnetic beads, and Cas nuclease effector protein. Each barcode sequence and guide sequence may preferably bind a specific target nucleic from the sample, such that the sample does not need to be divided into separate assays.

A dosimeter or badge may be provided with the device as described herein that serves as a sensor or indicator, such that the wearer may be notified of exposure to certain microbes or other agents. Providing such a dosimeter or badge with the device may be particularly useful for first responders, surveillance of soldiers or other military personnel, as well as clinicians, researchers, and hospital staff, in order to provide information relating to exposure to potentially dangerous agents as quickly as possible, for example for biological or chemical warfare agent detection. Such a surveillance badge may be used for preventing exposure to dangerous microbes (or pathogens) in, for example, immunocompromised patients, burn patients, patients undergoing chemotherapy, children, or elderly.

Near-real-time microbial diagnostics may be beneficial for food, clinical, industrial, and other environmental settings. Hence, the present invention may be used for rapid detection of, for example, foodborne pathogens, using one or more barcode sequences or guide sequences that are specific to one or more target pathogens.

Other embodiments described herein relate to a kit-of-parts for detecting an analyte, such as nucleic acids, which includes the magnetic beads, the barcode sequences, the guide sequences, the Cas nuclease effector proteins, and any additional reagents.

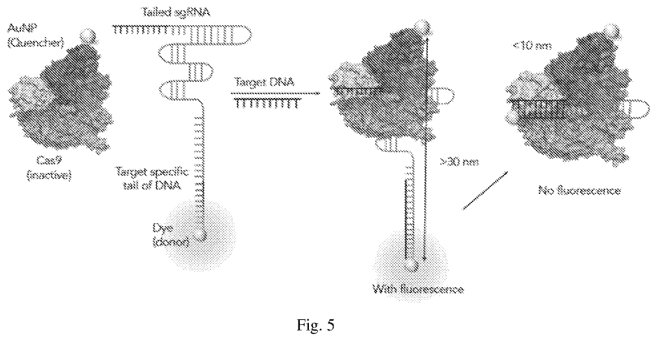

Still other embodiments described herein relate to CRISPR-based molecular beacon systems. As illustrated in FIG. 5, a CRISPR-Cas protein can be engineered with metallic nanoparticle acceptors and a tailed-guiding RNA (gRNA) terminally modified with donor fluorescent dyes to assemble in the presence of target DNA, forming a CRISPR complex with enhanced energy transfer and fluorescence quenching. The target DNA interacts with the tail of the gRNA molecules to form dsDNA that triggers its binding to CRISPR-Cas protein bringing the donor fluorescent dyes to the proximity of metal nanoparticle acceptors.

The invention is further illustrated by the following examples, which is not intended to limit the scope of the claims.

### Example 1

### CRISPR Barcoding Technology

This example describes a CRISPR-based approach that uses specifically designed DNA barcodes for the detection and testing of one or multiple targets. Each of the DNA barcodes comprises an ssDNA sequence that is complementary to the target gene sequence and terminally modified with specific small marker molecules.

We use these DNA barcodes to integrate the unique ability of CRISPR-Cas9 to recognize and cleave double-stranded DNA (dsDNA) sequences with magnetic separation, allowing for rapid detection of single-stranded DNA (ssDNA) target sequence without the need for extensive sample processing or thermocycling.

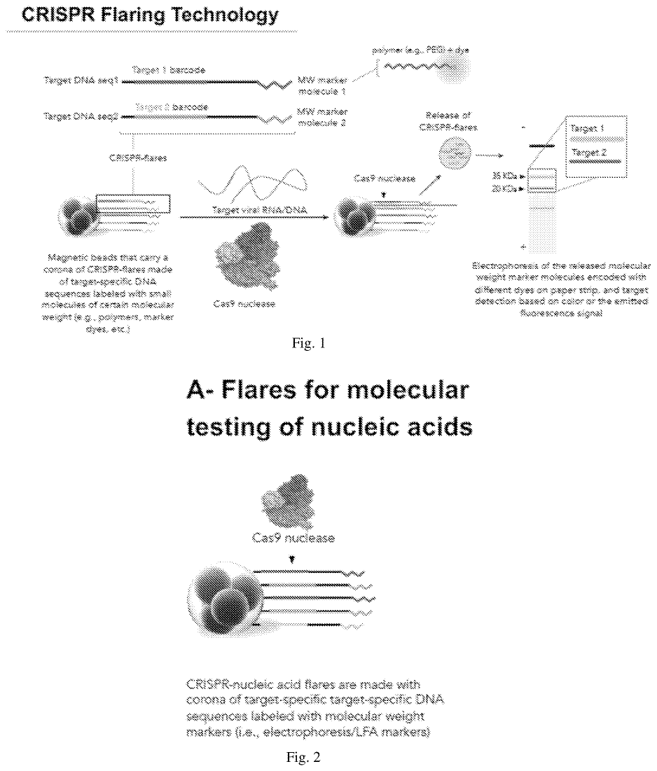

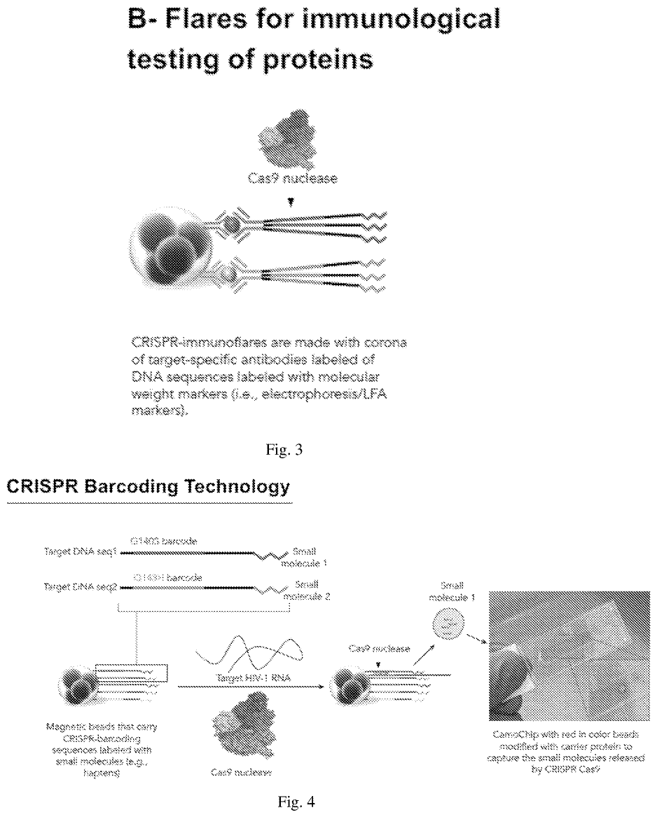

As illustrated in FIGS. 1-3, magnetic beads that carry corona made of ssDNA barcoding sequences terminally modified with detectable reporter molecules (e.g., fluorescent dyes, colorimetric dyes, marker molecules with specific molecular weight, electrical marker molecules, haptens, co-factors, nanoparticles) are mixed with the sample. In the presence of the target sequence, it binds to its complimentary barcode DNA forming a double-stranded DNA (dsDNA) on the surface of beads that can be specifically recognized and cleaved with the CRISPR-Cas9 system, which results in releasing the small reporter molecules in the reaction medium. Beads are separated with an external magnetic field and the released marker molecules can be detected with high sensitivity and specificity.

DNA barcodes can be designed to target one or multiple genes of the same organism or multiple organisms. DNA barcodes can be designed to test or monitor differences in nucleic sequences down to a single nucleotide. Beads can carry mono (of the same sequence) or mixed (of multiple sequences) DNA corona that targets one organism or multiple organisms.

Small marker molecules tethered to the barcoding DNA can be engineered to allow the integration of this platform with currently existing assays and different signal detection platforms, including, color detectors, turbidimeters, fluorometers, an electrical signal detector, lateral flow assay, electrophoresis, chromatography and magnetic readers.

Small marker molecules tethered to the barcoding DNA can be engineered to allow signal amplification. For instance: (i) the small molecule can be degradable nanoparticles that release a significant number of molecules in the medium after the CRISPR reaction, (ii) the small molecule can be a co-factor (enzyme subunit, fragment, co-enzyme) that activates enzyme to generate color, fluorescence, or breakdown a large substrate into detectable molecules, or (iii) the small molecule can be an easy to amplify DNA sequence.

### Methods

1—We use thiolated magnetic beads that are activated with 10 mM TCEP for 30 minutes at room temperature. Then washed with PBS pH 7.4 3 times.
2—We use DNA barcodes terminally modified with maleimide groups at the 5′ end and streptavidin at the 3′ end to prepare a mixed DNA corona on the surface of beads to detect target 1 and target 2.
3—DNA barcodes designed for target-1 are mixed with FITC-modified biotin and incubated for 30 minutes at room temperature to form barcodes carrying FITC green-fluorescent dye.
4—DNA barcodes designed for target-2 are mixed with Rhodamine-modified biotin and incubated for 30 minutes at room temperature to form barcodes carrying Rhodamine red-fluorescent dye.
5—A mixture of the two DNA barcodes is prepared at 1:1molar ratio.
6—The prepared mixture is then mixed with the activated thiolated beads in PBS pH 7.4 to allow coupling of beads thiol groups with the DNA barcode maleimide groups at a molar ratio of 1 bead: 100,000 DNA molecules (including 50% barcodes for target 1 and 50% barcodes for target 2). The reaction mixture is incubated for 2 hours at room temperature with shaking.
7—The unbound DNA and biotin are removed by 3-step washing using magnetic separation.
8—The prepared beads are then mixed with the target DNAs.
9 —CRISPR-Cas9 preassembled with gRNAs that target cleaving target 1 and target 2 sequences are added to the beads in the CRISPR reaction mixture from NEB, Inc., and incubated for 1 hour for complete digestion at room temperature.
10—Beads are magnetically separated and the released DNA-streptavidin that carries red or green fluorescent labeled biotin is detected in the supernatant using fluorescence microscopy or loaded for electrophoresis and detected on an LFA-like cartridge.

### Example 2

### CRISPR Barcoding Technology

In this example, we describe the ability of CRISPR-Cas9 to break double stranded nucleic acid made of barcoding sequences to small molecules detected on CamoChip, described in PCT/US2024/010511, filed Jan. 5, 2024, which is incorporated by reference in its entirety. As illustrated in FIG. 4, each barcoding sequence codes for a specific target DNA. The target DNA will bind to its complementary barcode sequence on the surface of magnetic beads. Magnetic beads are isolated then mixed with Cas9 systems in the nuclease buffer to release labels of small molecules. Small molecules are easy to detect and packed in a chain to amplify the presence of the target. The released small molecules made of haptens are then loaded on the surface of CamoChip, in which beads' surface are modified carrier protein to capture these different haptens for multiplex detection of the barcoded sequences on chip. Then PtNPs modified with antibodies that target these hapten/carrier protein complex is added to decamouflage the beads and allow its detection with optical sequence.

This technology provides a significant ability for multiplex testing of nucleic acid with the need of amplification and highly reduced cost as the hapten/protein pairing is well established and of low cost. It can allow cancer screening by detecting mutations, SNP, drug resistance and many genetic disorders, sickle cell diseases and so on.

### Example 3

### CRISPR Beacons for Molecular Detection and Genotyping

In this example, we describe a CRISPR-based molecular beacon systems. As illustrated in FIG. 5, we rely on using a CRISPR system comprised of CRISPR-Cas protein specifically engineered with metallic nanoparticle acceptors and a tailed-guiding RNA (gRNA) terminally modified with fluorescent dyes donors to assemble in the presence of target DNA, forming a CRISPR complex with enhanced energy transfer and fluorescence quenching. The target DNA interacts with the tail of the gRNA molecules to form dsDNA that triggers its binding to CRISPR-Cas protein bringing the donor fluorescent dyes to the proximity of metal nanoparticle acceptors. The coherent plasmonic resonance between the metal and fluorescent dyes is exploited to achieve improved donor-acceptor resonance within the gut of CRISPR-Cas that is around 10 nm in size. This class of beacons combines the specificity of CRISPR to detect nucleic acid sequences with the efficiency of nanoparticles as quenchers to provide high detection specificity and sensitivity. The design is flexible and can be engineered to allow for single and multiplex detection of various targets, including viruses, bacteria, other microbes, cancer, and even evaluation of gene expressions.

From the above description of the invention, those skilled in the art will perceive improvements, changes and modifications. Such improvements, changes and modifications within the skill of the art are intended to be covered by the appended claims. All references, publications, and patents cited in the present application are herein incorporated by reference in their entirety.

1. A system for detecting an analyte in a sample, the system comprising:
a plurality of magnetic beads and a plurality of analyte capture molecules extending from the surfaces of the beads;
a plurality of barcode sequences that are configured to specifically bind to the analyte in the sample, each barcode sequence including a nucleic acid and a reporter molecule, wherein the nucleic acid of the barcode sequence is configured to be cleaved from the barcode sequence by a Cas nuclease effector protein after binding of the barcode sequence to the analyte; and
a Cas nuclease configured to cleave the nucleic acid and release the reporter molecule after binding of the barcode sequences with the analyte.

2. The system of claim 1, wherein the analyte is a targeted nucleic acid in the sample and the analyte capture molecules include and/or define the barcode sequences and wherein each of the barcode sequence includes a single-stranded target-specific nucleic acid sequence that is complementary to or hybridizes with at least a portion of a targeted nucleic acid and a reporter molecule that is configured to be cleaved from the barcode sequence by a Cas nuclease effector protein after binding of target-specific nucleic acid to the at least portion of the targeted nucleic acid.

3. The system of claim 2, wherein the Cas nuclease effector protein is complexed with a guide RNA sequence that includes a nucleotide sequence that hybridizes to or is complementary to the target-specific nucleic acid sequence or the portion of the targeted nucleic acid.

4. The system of claim 2, wherein the target-specific nucleic acid sequence of the barcode sequences includes a first end linked to the magnetic bead and a second terminal end linked to the reporter molecule.

5. (canceled)

6. The system of claim 1, wherein the reporter molecule includes a small molecule that is degradable upon cleavage of the reporter molecule to release a plurality of detectable molecules indicative of the cleavage; or
the reporter molecule is a co-factor that activates an enzyme to generate color, fluorescence, or breakdown of a substrate into a detectable molecule; or
the reporter molecule includes a DNA sequence that is readily amplified.

7. (canceled)

8. (canceled)

9. The system of claim 1, wherein the Cas nuclease effector protein comprises a Cas9, Cas12, or Cas13 nuclease.

10. The system of claim 1, wherein the target-specific nucleic acid of the barcode sequence is configured to target one or multiple genes of an organism or multiple organisms.

11. The system of claim 1, wherein the plurality of barcode sequences include: first barcode sequences that include first target-specific nucleic acids and first reporter molecules; and second barcode sequences that include second target-specific nucleic acids and second reporter molecules that differ from the first target-specific nucleic acids and first reporter molecules.

12. The system of claim 11, wherein the Cas nuclease effector protein includes a first Cas nuclease effector protein complexed with first guide RNA that is complementary or hybridizes to the first target specific nucleic acids or a first targeted nucleic acid and a second Cas nuclease effector protein complexed with a second guide RNA that is complementary or hybridizes to the second target-specific nucleic acids or a second targeted nucleic acid.

13. The system of claim 1, wherein the plurality of barcode sequences include barcode sequence having differing target specific nucleic acids and reporter molecules and the Cas nuclease effector protein includes Cas nuclease effector protein with differing guide RNAs that are complementary or hybridize to the differing target-specific nucleic acids or differing targeted nucleic acids.

14. The system of claim 1, wherein the plurality of barcode sequences are directly or indirectly linked to a plurality of plurality of magnetic beads.

15. The system of claim 14, wherein the plurality of barcode sequences are topographically and/or spatially arranged on an outer surface of the magnetic beads.

16. The system of claim 14, wherein the plurality of barcode sequences are linked to the magnetic beads using a click reaction chemistry.

17. The system of claim 1, wherein the analyte is a targeted polypeptide in the sample and the plurality of barcode sequences include a nucleic acid with a first end and a second end, the first end being linked to an antibody or antigen binding fragment thereof that specifically binds to the polypeptide and the second end includes a reporter molecule, wherein the nucleic acid of the barcode sequence is configured to be cleaved from the barcode sequence by the Cas nuclease effector protein after binding of barcode sequence to the polypeptide.

18. The system of claim 17, wherein the Cas nuclease effector protein is complexed with a guide RNA sequence that includes nucleotide sequence that hybridizes or is complementary to at least a portion of the nucleic acid of the barcode sequence.

19. (canceled)

20. The system of claim 17, wherein the reporter molecule includes a small molecule that is degradable upon cleavage of the reporter molecule to release a plurality of detectable molecules indicative of the cleavage; or
the reporter molecule is a co-factor that activates an enzyme to generate color, fluorescence, or breakdown of a substrate into a detectable molecule; or
the reporter molecule includes a DNA sequence that is readily amplified.

21. (canceled)

22. (canceled)

23. The system of claim 17, wherein the Cas nuclease effector protein comprises a Cas9, Cas12, or Cas13 nuclease effector protein.

24. The system of claim 17, further comprising a plurality of magnetic beads and a plurality of second antibody or antigen binding fragments thereof directly or indirectly linked to the beads, the second antibody or antigen binding fragments thereof specifically binding the peptide or protein.

25. The system of claim 24, wherein the plurality of second antibodies or antigen binding fragments thereof are topographically and/or spatially arranged on an outer surface of the magnetic beads.

26. The system of claim 24, wherein the second antibody or antigen binding fragment thereof binds to a different epitope of the polypeptide than the first antibody or antigen binding fragment thereof of the barcode sequence.

27-46. (canceled)
